(19) United States
(12) Patent Application Publication
(10) Pub. No.: US 2006/0205719 A1
(43) Pub. Date: Sep. 14, 2006

(54) Novel compounds having an antibacterial activity

(75) Inventors: Christian Hubschwerlen, Durmenach (FR); Jean P Surivet, Saint-Louis (FR); Cornelia Zumbrunn, Basel (CH)

Correspondence Address: EDWARDS & ANGELL, LLP, P.O. BOX 55874, BOSTON, MA, 02205, US

(73) Assignee: Morphochem Aktiengesellschaft Fur, Munchen (DE)

(21) Appl. No.: 10/553,731

(22) PCT Filed: Mar. 29, 2004
(86) PCT No.: PCT/EP04/03306

§ 371 Date: Jan. 31, 2006

(30) Foreign Application Priority Data
Apr. 8, 2003 (DE) 103 16 081.7

(51) Int. Cl. A61K 31/538; A61K 31/355; C07D 265/36; C07D 319/14

(52) U.S. Cl. 514/230.5; 544/105; 514/452; 549/362

(57) ABSTRACT

The present invention describes novel anti-bacterial compounds of formula (I). 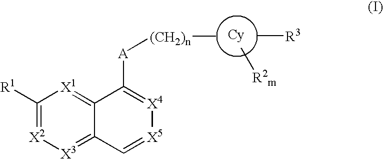 These compounds are, amongst others, of interest as inhibitors of Topoisomerase IV (Topo IV) as well as of DNA gyrase.

Resistance to the antibiotics used currently has increased appreciably in many countries of the world in recent years and in some cases has assumed alarming proportions. The main problem is that those pathogens exhibit not just a single resistance but, as a rule, multiple resistance. This is true especially for some gram-positive pathogen groups, such as staphylococci, pneumococci and enterococci (S. Ewig et al., Antibiotika-Resistenz bei Erregern ambulant erworbener Atemwegsinfektionen (Antibiotic resistance in pathogens of outpatient-acquired respiratory tract infections), Chemother. J. 2002, 11, 12-26; F. Tenover, Development and spread of bacterial resistance to antimicrobial agents: an overview, Clin. Infect. Dis. 2001 Sep 15, 33 Suppl. 3, 108-115).

A long-feared development has recently occurred: In the USA, the first strain of Staphylococcus aureus has been described that is not only resistant to methicillin but also highly resistant to vancomycin (Centers for Disease Control and Prevention, Staphylococcus aureus resistant to vancomycin—United States, 2002, MMWR 2002, 51, 565-567). In addition to hygiene measures in hospitals, therefore, increased efforts are also required to find new antibiotics that as far as possible have a novel structure and a novel mechanism of action so as to be effective against those problem bacteria.

The present invention describes new kinds of compounds having anti-bacterial activity. These compounds are, amongst others, of interest as inhibitors of Topoisomerase IV (Topo IV) as well as of DNA gyrase.

The present invention relates to compounds of the general formula (I): 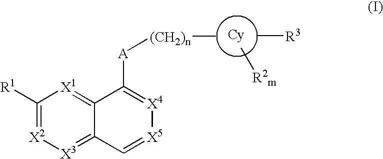
wherein
A is an oxygen or a sulphur atom, a NH, an alkylene, an alkenylene, an alkynylene or a heteroalkylene group,
X¹, X², X³, X⁴ and X⁵ are each independently of the others nitrogen atoms or groups of formula CH or CR
Cy is a cycloalkylene, a heterocycloalkylene, an arylene or a heteroarylene group,
R¹ is a hydrogen atom, a halogen atom, a hydroxy, an amino, a mercapto, an alkyl, a heteroalkyl, an alkyloxy, a heteroalkyloxy, a cycloalkyl, a heterocycloalkyl, an alkylcycloalkyl, a heteroalkylcycloalkyl, a cycloalkyloxy, an alkylcycloalkyloxy, a hetero-cycloalkyloxy or a heteroalkylcycloalkyloxy group,
the radicals R², each independently of any other(s), are a halogen atom, a hydroxy, an amino, a nitro or a mercapto group, an alkyl, an alkenyl, an alkynyl, a heteroalkyl, an aryl, a heteroaryl, a cycloalkyl, an alkylcycloalkyl, a heteroalkylcycloalkyl, a heterocycloalkyl, an aralkyl or a heteroaralkyl radical, or two of the radicals R² together form part of an aryl, heteroaryl, cycloalkyl, heterocycloalkyl, alkylcycloalkyl, heteroalkylcycloalkyl, aralkyl or a heteroaralkyl ring system,
R³ is an alkyl, alkenyl, alkynyl, heteroalkyl, aryl, heteroaryl, cycloalkyl, alkylcycloalkyl, heteroalkyl-cycloalkyl, heterocycloalkyl, aralkyl or heteroaralkyl radical,
R⁴ is a halogen atom, or a hydroxy, alkyl, alkenyl, alkynyl or heteroalkyl group,
n is 0, 1 or 2, and
m is 0, 1 or 2,
or a pharmacologically acceptable salt, solvate, hydrate or a pharmacologically acceptable formulation thereof.

The expression alkyl refers to a saturated, straight-chain or branched hydrocarbon group that contains from 1 to 20 carbon atoms, preferably from 1 to 12 carbon atoms, especially from 1 to 6 carbon atoms, for example a methyl, ethyl, propyl, iso-propyl, n-butyl, iso-butyl, tert-butyl, n-pentyl, n-hexyl, 2,2-dimethylbutyl or n-octyl group.

The expressions alkenyl and alkynyl refer to at least partially unsaturated, straight-chain or branched hydrocarbon groups that contain from 2 to 20 carbon atoms, preferably from 2 to 12 carbon atoms, especially from 2 to 6 carbon atoms, for example an ethenyl, allyl, acetylenyl, propargyl, isoprenyl or hex-2-enyl group. Preferably, alkenyl groups have one or two (especially one) double bond(s) and alkynyl groups have one or two (especially one) triple bond(s).

Furthermore, the terms alkyl, alkenyl and alkynyl refer to groups in which one or more hydrogen atoms have been replaced by a halogen atom (preferably F or Cl) such as, for example, a 2,2,2-trichloroethyl or a trifluoromethyl group.

The expression heteroalkyl refers to an alkyl, alkenyl or alkynyl group in which one or more (preferably 1, 2 or 3) carbon atoms have been replaced by an oxygen, nitrogen, phosphorus, boron, selenium, silicon or sulphur atom (preferably oxygen, sulphur or nitrogen). The expression heteroalkyl furthermore refers to a carboxylic acid or to a group derived from a carboxylic acid such as, for example, acyl, acylalkyl, alkoxycarbonyl, acyloxy, acyl-oxyalkyl, carboxyalkylamide or alkoxycarbonyloxy.

Examples of heteroalkyl groups are groups of formulae R^{a}—O—Y^{a}—, R^{a}—S—Y^{a}—, R^{a}—N(R^{b})—Y^{a}—, R^{a}—CO—Y^{a}—, R^{a}—O—CO—Y^{a}—, R^{a}—CO—O—Y^{a}—, R^{a}—CO—N(R^{b})—Y^{a}—, R^{a}—N(R^{b})—CO—Y^{a}—, R^{a}—O—CO—N(R^{b})—Y^{a}—, R^{a}—B(R^{b})—CO—O—Y^{a}, R^{a}—N(R^{b})—CO—N(R^{c})—Y^{a}—, R^{a}—O—CO—O—Y^{a}—, R^{a}—N(R^{b})—C(═NR^{d})—N(R^{c})—Y^{a}—, R^{a}—CS—Y^{a}, R^{a}—O—CS—Y^{a}, R^{a}—CS—O—Y^{a}, R^{a}—CS—N(R^{b})—Y^{a}—, R^{a}—N(R^{b})—CS—Y^{a}—, R^{a}—O—CS—N(R^{b})—Y^{a}—, R^{a}—N(R^{b})—CS—O—Y^{a}, R^{a}—N(R^{b})—CS—N(R^{c})—Y^{a}—, R^{a}—O—CS—O—Y^{a}, R^{a}—S—CO—Y^{a}, R^{a}—CO—S—Y^{a}, R^{a}—S—CO—N(R^{b})—Y^{a}—, R^{a}—N(R^{b})—CO—S—Y^{a}—, R^{a}—S—CO—O—Y^{a}, R^{a}—O—CO—S—Y^{a}—, R^{a}—S—CO—S—Y^{a}, R^{a}—S—CS—Y^{a}—, R^{a}—CS—S—Y^{a}—, R^{a}—S—CS—N(R^{b})—Y^{a}—, R^{a}—N(R^{b})—CS—S—Y^{a}—, R^{a}—S—CS—O—Y^{a}—, R^{a}—O—CS—S—Y^{a}—, R^{a} being a hydrogen atom, a C₁-C₆alkyl, a C₂-C₆alkenyl or a C₂-C₆alkynyl group; R^{b} being a hydrogen atom, a C₁-C₆alkyl, a C₂-C₆alkenyl or a C₂-C₆alkynyl group; RC being a hydrogen atom, a C₁-C₆alkyl, a C₂-C₆alkenyl or a C₂-C₆alkynyl group; Rd being a hydrogen atom, a C₁-C₆alkyl, a C₂-C₆alkenyl or a C₂-C₆alkynyl group and Y^{a} being a direct bond, a C₁-C₆alkylene, a C₂-C₆alkenylene or a C₂-C₆alkynylene group, each heteroalkyl group containing at least one carbon atom and it being possible for one or more hydrogen atoms to have been replaced by fluorine or chlorine atoms. Specific examples of heteroalkyl groups are methoxy, trifluoromethoxy, ethoxy, n-propyloxy, isopropyloxy, tert-butyloxy, methoxymethyl, ethoxymethyl, methoxyethyl, methylamino, ethylamino, dimethylamino, diethylamino, isopropylethylamino, methylaminomethyl, ethylaminomethyl, diisopropylaminoethyl, enol ether, dimethylaminomethyl, dimethylaminoethyl, acetyl, propionyl, butyryloxy, acetyloxy, methoxycarbonyl, ethoxycarbonyl, N-ethyl-N-methylcarbamoyl and N-methylcarbamoyl. Further examples of heteroalkyl groups are nitrile, isonitrile, cyanate, thiocyanate, isocyanate, isothiocyanate and alkylnitrile groups.

The expression cycloalkyl refers to a saturated or partially unsaturated (for example cycloalkenyl), cyclic group that contains one or more rings (preferably 1 or 2), which build a scaffold containing from 3 to 14 carbon atoms, preferably from 3 to 10 (especially 3, 4, 5, 6 or 7) carbon atoms. The expression cycloalkyl refers furthermore to groups in which one or more hydrogen atoms have been replaced by fluorine, chlorine, bromine or iodine atoms or by OH, ═O, SH, ═S, NH₂, ═NH or NO₂ groups, thus, for example, cyclic ketones such as, for example, cyclohexanone, 2-cyclohexenone or cyclopentanone. Further specific examples of cycloalkyl groups are a cyclopropyl, cyclobutyl, cyclopentyl, spiro[4,5]decanyl, norbornyl, cyclohexyl, cyclopentenyl, cyclohexadienyl, decalinyl, bicyclo[4.3.0]nonyl, tetralin, cyclopentylcyclohexyl, fluorocyclohexyl or cyclohex-2-enyl group.

The expression heterocycloalkyl refers to a cycloalkyl group as defined above in which one or more (preferably 1, 2 or 3) ring carbon atoms have been replaced by an oxygen, nitrogen, silicon, selenium, phosphorus or sulphur atom (preferably oxygen, sulphur or nitrogen). A heterocycloalkyl group has preferably 1 or 2 ring(s) containing from 3 to 10 (especially 3, 4, 5, 6 or 7) ring atoms. The expression heterocycloalkyl refers furthermore to groups in which one or more hydrogen atoms have been replaced by fluorine, chlorine, bromine or iodine atoms or by OH, ═O, SH, ═S, NH₂, ═NH or NO₂ groups. Examples are a piperidyl, piperazinyl, morpholinyl, urotropinyl, pyrrolidinyl, tetrahydrothiophenyl, tetrahydropyranyl, tetrahydrofuryl or 2-pyrazolinyl group and also lactams, lactones, cyclic imides and cyclic anhydrides.

The expression alkylcycloalkyl refers to groups containing both cycloalkyl and also alkyl, alkenyl or alkynyl groups in accordance with the above definitions, for example alkylcycloalkyl, cycloalkylalkyl, alkylcycloalkenyl, alkenylcycloalkyl and alkynylcyclo-alkyl groups. An alkylcycloalkyl group preferably contains a cycloalkyl group that contains one or two ring systems which build a scaffold containing from 3 to 10 (especially 3, 4, 5, 6 or 7) carbon atoms, and one or two alkyl, alkenyl or alkynyl groups having 1 or 2 to 6 carbon atoms.

The expression heteroalkylcycloalkyl refers to alkylcycloalkyl groups as defined above in which one or more (preferably 1, 2 or 3) carbon atoms have been replaced by an oxygen, nitrogen, silicon, selenium, phosphorus or sulphur atom (preferably oxygen, sulphur or nitrogen). A heteroalkylcycloalkyl group preferably contains 1 or 2 ring systems having from 3 to 10 (especially 3, 4, 5, 6 or 7) ring atoms, and one or two alkyl, alkenyl, alkynyl or heteroalkyl groups having from 1 or 2 to 6 carbon atoms. Examples of such groups are alkylheterocycloalkyl, alkylheterocycloalkenyl, alkenyl-heterocycloalkyl, alkynylheterocycloalkyl, hetero-alkylcycloalkyl, heteroalkylheterocycloalkyl and hetero-alkylheterocycloalkenyl, the cyclic groups being saturated or mono-, di- or tri-unsaturated.

The expression aryl or Ar refers to an aromatic group that has one or more rings and that is formed by a scaffold containing from 6 to 14 carbon atoms, preferably from 6 to 10 (especially 6) carbon atoms. The expression aryl (or Ar) refers furthermore to groups in which one or more hydrogen atoms have been replaced by fluorine, chlorine, bromine or iodine atoms or by OH, SH, NH₂ or NO₂ groups. Examples are a phenyl, naphthyl, biphenyl, 2-fluorophenyl, anilinyl, 3-nitrophenyl or 4-hydroxyphenyl group.

The expression heteroaryl refers to an aromatic group that has one or more rings and is formed by a scaffold containing from 5 to 14 ring atoms, preferably from 5 to 10 (especially 5 or 6) ring atoms, and contains one or more (preferably 1, 2, 3 or 4) oxygen, nitrogen, phosphorus or sulphur ring atoms (preferably O, S or N). The expression heteroaryl refers furthermore to groups in which one or more hydrogen atoms have been replaced by fluorine, chlorine, bromine or iodine atoms or by OH, SH, NH₂ or NO₂ groups. Examples are 4-pyridyl, 2-imidazolyl, 3-phenylpyrrolyl, thiazolyl, oxazolyl, triazolyl, tetrazolyl, isoxazolyl, indazolyl, indolyl, benzimidazolyl, pyridazinyl, quinolinyl, purinyl, carbazolyl, acridinyl, pyrimidyl, 2,3′-bifuryl, 3-pyrazolyl and isoquinolinyl groups.

The expression aralkyl refers to groups containing both aryl and also alkyl, alkenyl, alkynyl and/or cycloalkyl groups in accordance with the above definitions, such as, for example, arylalkyl, arylalkenyl, arylalkynyl, arylcycloalkyl, arylcycloalkenyl, alkylarylcycloalkyl and alkylarylcycloalkenyl groups. Specific examples of aralkyls are toluene, xylene, mesitylene, styrene, benzyl chloride, o-fluorotoluene, 1H-indene, tetralin, dihydro-naphthalene, indanone, phenylcyclopentyl, cumene, cyclo-hexylphenyl, fluorene and indan. An aralkyl group preferably contains one or two aromatic ring systems (1 or 2 rings) containing from 6 to 10 carbon atoms and one or two alkyl, alkenyl and/or alkynyl groups containing from 1 or 2 to 6 carbon atoms and/or a cycloalkyl group containing 5 or 6 ring carbon atoms.

The expression heteroaralkyl refers to an aralkyl group as defined above in which one or more (preferably 1, 2, 3 or 4) carbon atoms have been replaced by an oxygen, nitrogen, silicon, selenium, phosphorus, boron or sulphur atom (preferably oxygen, sulphur or nitrogen), that is to say to groups containing both aryl or heteroaryl and also alkyl, alkenyl, alkynyl and/or heteroalkyl and/or cycloalkyl and/or heterocycloalkyl groups in accordance with the above definitions. A heteroaralkyl group preferably contains one or two aromatic ring systems (1 or 2 rings) containing from 5 or 6 to 10 carbon atoms and one or two alkyl, alkenyl and/or alkynyl groups containing 1 or 2 to 6 carbon atoms and/or a cycloalkyl group containing 5 or 6 ring carbon atoms, 1, 2, 3 or 4 of those carbon atoms having been replaced by oxygen, sulphur or nitrogen atoms.

Examples are arylheteroalkyl, arylheterocycloalkyl, aryl-heterocycloalkenyl, arylalkylheterocycloalkyl, arylalkenylheterocycloalkyl, arylalkynylheterocycloalkyl, arylalkylheterocycloalkenyl, heteroarylalkyl, heteroaryl-alkenyl, heteroarylalkynyl, heteroarylheteroalkyl, heteroarylcycloalkyl, heteroarylcycloalkenyl, heteroaryl-heterocycloalkyl, heteroarylheterocycloalkenyl, hetero-arylalkylcycloalkyl, heteroarylalkylheterocycloalkenyl, heteroarylheteroalkylcycloalkyl, heteroarylheteroalkyl-cycloalkenyl and heteroarylheteroalkylheterocycloalkyl groups, the cyclic groups being saturated or mono-, di- or tri-unsaturated. Specific examples are a tetrahydro-isoquinolinyl, benzoyl, 2- or 3-ethylindolyl, 4-methyl-pyridino, 2-, 3- or 4-methoxyphenyl, 4-ethoxyphenyl, 2-, 3- or 4-carboxyphenylalkyl group.

The expressions cycloalkyl, heterocycloalkyl, alkylcyclo-alkyl, heteroalkylcycloalkyl, aryl, heteroaryl, aralkyl and heteroaralkyl refer to groups in which one or more hydrogen atoms of such groups have been replaced by fluorine, chlorine, bromine or iodine atoms or by OH, ═O, SH, ═S, NH₂, ═NH or NO₂ groups.

The expression “optionally substituted” refers to groups in which one or more hydrogen atoms have been replaced by fluorine, chlorine, bromine or iodine atoms or by OH, ═O, SH, ═S, NH₂, ═NH or NO₂ groups. This expression refers furthermore to groups that are substituted by unsubstituted C₁-C₆alkyl, C₂-C₆alkenyl, C₂-C₆alkynyl, C₁-C₆heteroalkyl, C₃-C₁₀cycloalkyl, C₂-Cgheterocycloalkyl, C₆-C₁₀aryl, C₁-C₉heteroaryl, C₇-C₁₂aralkyl or C₂-C₁₁heteroaralkyl groups.

Owing to their substitution, compounds of formula (I) may contain one or more centres of chirality. The present invention therefore includes both all pure enantiomers and all pure diastereoisomers and also mixtures thereof in any mixing ratio. The present invention moreover also includes all cis/trans-isomers of the compounds of the general formula (I) and also mixtures thereof. The present invention moreover includes all tautomeric forms of the compounds of formula (I).

Preferred are compounds of formula (I) wherein A is an oxygen or a sulphur atom or a group of formula CH₂, CH₂CH₂, CH₂N(C₁-C₄alkyl), N(C₁-C₄alkyl)CH₂, CH₂O, OCH₂, CH₂S, SCH₂, CH₂CH(OH), CH(OH), CH(OH)CH₂, NHCO, CONH, C(═O)CH₂ or CH₂C(═O).

Also preferred are compounds of formula (I) wherein three, four or five of the groups X¹, X², X³, X⁴ and X⁵ are CH groups.

Further preferred is R¹ a C₁-C₄alkyloxy or a C₁-C₄hetero-alkyloxy group, wherein one or more hydrogen atoms of such groups may have been replaced by fluorine atoms.

Especially preferred is R¹ a methoxy group.

Also preferred is R² a hydroxy, a C₁-C₄alkyl, a C₁-C₄heteroalkyl or a C₆-C₁₂heteroaralkyl group.

Furthermore preferably, R³ is a heteroalkylcycloalkyl or a heteroaralkyl group.

R³ is especially preferably a group of formula -B-Y, wherein B is an alkylene (especially a C₁-C₄alkylene group), an alkenylene, an alkynylene or a heteroalkylene group (especially a C₁-C₄heteroalkylene group) and Y is an aryl, a heteroaryl, an aralkyl, a heteroaralkyl, a cycloalkyl, a heterocycloalkyl, an alkylcycloalkyl or a heteroalkylcycloalkyl group (especially a heterocycloalkyl or an arylheterocyloalkyl group). Furthermore, Y has preferably one of the following structures: 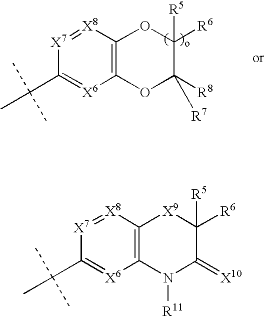
wherein X⁶, X⁷ and X⁸ are each independently of the others nitrogen atoms or groups of formula CR⁹, X⁹ and X¹⁰ are each independently of the others oxygen or sulphur atoms or groups of formula NR¹⁰, o is 0, 1 or 2, R⁵, R⁶, R⁷, R⁸ and R⁹ are each independently of the others hydrogen atoms, halogen atoms, hydroxy, alkyl, alkenyl, alkynyl or heteroalkyl groups and R¹⁰ and R¹¹ are each independently of the others hydrogen atoms, alkyl, alkenyl, alkynyl or heteroalkyl groups.

Especially preferably, Y has one of the following structures:

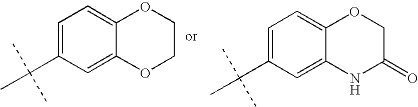

Also preferred the linker -A-(CH₂)ₙ— has a chain length of 2 or 3 atoms.

Furthermore preferred R⁴ is a fluorine or a chlorine atom or a C₁-C₄alkyloxy or a C₃-C₆dialkylaminomethyl group wherein one or more hydrogen atoms of such groups may have been replaced by fluorine atoms.

Also preferably Cy is a cycloalkylene or a heterocyclo-alkylene group containing one or two rings and 4, 5, 6, 7, 8, 9 or 10 ring atoms.

Especially preferred Cy is a group of formulas 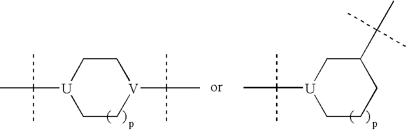
wherein U is a nitrogen atom or a group of formula CH or COH and V is a nitrogen atom or a CH group and p is 0 or 1. The substituents respectively may be bonded equatorially as well as axially to these groups.

The therapeutic use of compounds of formula (I), their pharmacologically acceptable salts or solvates and hydrates and also formulations and pharmaceutical compositions also lie within the scope of the present invention.

The pharmaceutically compositions according to the present invention comprise at least one compound of formula (I) as active ingredient and, optionally, carrier substances and/or adjuvants.

Examples of pharmacologically acceptable salts of the compounds of formula (I) are salts of physiologically acceptable mineral acids, such as hydrochloric acid, sul-phuric acid and phosphoric acid, or salts of organic acids, such as methanesulphonic acid, p-toluenesulphonic acid, lactic acid, acetic acid, trifluoroacetic acid, citric acid, succinic acid, fumaric acid, maleic acid and salicylic acid. Further examples of pharmacologically acceptable salts of the compounds of formula (I) are alkali metal and alkaline earth metal salts such as, for example, sodium, potassium, lithium, calcium or magnesium salts, ammonium salts or salts of organic bases such as, for example, methylamine, dimethylamine, triethylamine, piperidine, ethylenediamine, lysine, choline hydroxide, meglumine, morpholine or arginine salts. Compounds of formula (I) may be solvated, especially hydrated. The hydration may take place, for example, during the preparation process or as a consequence of the hygroscopic nature of the initially anhydrous compounds of formula (I). When the compounds of formula (I) comprise asymmetric C-atoms, they may be present either in the form of achiral compounds, diastereoisomeric mixtures, mixtures of enantiomers or in the form of optically pure compounds.

The pro-drugs to which the present invention also relates consist of a compound of formula (I) and at least one pharmacologically acceptable protecting group which will be removed under physiological conditions, such as, for example, an alkoxy-, aralkyloxy-, acyl- or acyloxy group, such as, for example, an ethoxy, benzyloxy, acetyl or acetyloxy group.

The present invention relates also to the use of those active ingredients in the preparation of medicaments. In general, compounds of formula (I) are administered either individually, or in combination with any other desired therapeutic agent, using the known and acceptable methods. Such therapeutically useful agents may be administered, for example, by one of the following routes: orally, for example in the form of dragees, coated tablets, pills, semi-solid substances, soft or hard capsules, solutions, emulsions or suspensions; parenterally, for example in the form of an injectable solution; rectally in the form of suppositories; by inhalation, for example in the form of a powder formulation or a spray; transdermally or intranasally. For the preparation of such tablets, pills, semi-solid substances, coated tablets, dragees and hard gelatine capsules, the therapeutically usable product may be mixed with pharmacologically inert, inorganic or organic pharmaceutical carrier substances, for example with lactose, sucrose, glucose, gelatine, malt, silica gel, starch or derivatives thereof, talcum, stearic acid or salts thereof, skimmed milk powder, and the like. For the preparation of soft capsules, pharmaceutical carrier substances such as, for example, vegetable oils, petroleum, animal or synthetic oils, wax, fat and polyols may be used. For the preparation of liquid solutions and syrups, pharmaceutical carrier substances such as, for example, water, alcohols, aqueous saline solution, aqueous dextrose, polyols, glycerol, vegetable oils, petroleum and animal or synthetic oils may be used. For suppositories, pharmaceutical carrier substances such as, for example, vegetable oils, petroleum, animal or synthetic oils, wax, fat and polyols may be used. For aerosol formulations, compressed gases that are suitable for this purpose, such as, for example, oxygen, nitrogen and carbon dioxide may be used. The pharmaceutically acceptable agents may also comprise additives for preserving and stabilising, emulsifiers, sweeteners, flavourings, salts for altering the osmotic pressure, buffers, encapsulation additives and antioxidants.

Combinations with other therapeutic agents may comprise other antimicrobial and anti-fungal active ingredients.

For the prevention and/or treatment of the above described diseases, the dose of the biologically active compound according to the invention may vary within wide limits and may be adjusted to individual requirements. Generally, a dose of from 10 mg to 4000 mg per day is suitable, a preferred dose being from 50 to 3000 mg per day. In suitable cases, the dose may also be below or above the stated values. The daily dose may be administered as a single dose or in a plurality of doses. A typical individual dose contains approximately 50 mg, 100 mg, 250 mg, 500 mg, 1 g or 2 g of the active ingredient.

### EXAMPLES

### Example 1

### (R,S)-6-{1-Hydroxy-2-[4-(7-methoxy-naphthalen-1-yloxymethyl)-piperidin-1-yl]-ethyl}-4H-benzo[1,4]oxazin-3-one

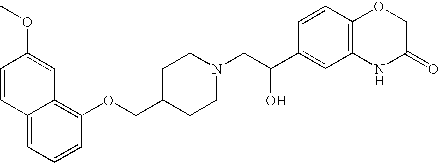

### Synthesis of 4-(7-methoxy-naphthalen-1-yloxymethyl)-piperidin-1-carboxylic acid tert-butyl ester

Diethylazodicarboxylate (755 mg, 4.3 mmol) was added dropwise to a solution of triphenylphosphine (1.14 g 4.3 mmol) in THF (5 ml). 4-Hydroxymethyl-piperidine-1-carboxylic acid tert-butyl ester (850 mg, 3.95 mmol) was added, followed by 7-methoxy-1-naphthol (synthesised according to Aust. J. Chem. 1993, 46, 731) (668 mg, 3.95 mmol). The yellow solution was stirred over night at room temperature, then concentrated and purified by column chromatography on silica gel (hexane/ethyl acetate 4:1) to give 1.11 g (76%) of a colourless oil.

MS (ESI+): 372.3 [M+H⁺]

### Synthesis of 4-(7-methoxy-naphthalen-1-yloxymethyl)-piperidine

Trifluoroacetic acid (2 ml) was added to a solution of 4-(7-Methoxy-naphthalen-1-yloxymethyl)-piperidine-1-carboxylic acid tert-butyl ester (1.11 g) in dichloromethane (10 ml) at room temperature under argon and stirred for two hours. The reaction mixture was concentrated by rotary evaporation, diluted in dichloromethane and washed with conc. ammonia. The organic layer was dried over MgSO₄ and concentrated.

### Synthesis of 6-{2-[4-(7-methoxy-naphthalen-1-yloxymethyl)-piperidin-1-yl]-acetyl}-4H-benzo[1,4]oxazin-3-one

Triethylamine (1 ml) was added to a mixture of 4-(7-methoxy-naphthalen-1-yloxymethyl)-piperidine (271 mg, 1 mmol) and 6-(2-chloro-acetyl)-4H-benzo[1,4]oxazin-3-one (225 mg, 1 mmol) in THF (5 ml) and stirred for 2 hours at 50° C. The reaction mixture was poured onto water and extracted with ethyl acetate. The organic layer was washed with NH₄Cl solution, dried over MgSO₄ and concentrated. The crystalline residue was stirred in methanol and ethyl acetate and filtered to give 250 mg (52%) of the pure product.

MS (ESI+) 461 [M+H⁺]

### Synthesis of (R,S)-6-{1-hydroxy-2-[4-(7-methoxy-naphthalen-1-yloxymethyl)-piperidin-1-yl]-ethyl}-4H-benzo[1,4]oxazin-3-one

NaBH₄ (1 eq) was added to a solution of 6-{2-[4-(7-methoxy-naphthalen-1-yloxymethyl)-piperidin-1-yl]-acetyl}-4H-benzo[1,4]oxazin-3-one (150 mg) in ethanol (2 ml) and stirred for 2 hours at room temperature. The reaction mixture was concentrated, diluted in water and the white crystals were filtered and dried under high vacuum to give 140 mg of the pure product.

MS (ESI+) 463.5 [M+H⁺]

### Example 2

### (R,S)-1-(2,3-Dihydro-benzo[1,4]dioxin-6-yl)-2-[4-(7-methoxy-naphthalen-1-yloxymethyl)-piperidin-1-yl]-ethanol

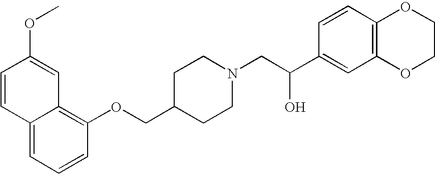

### Synthesis of 6-oxiranyl-2,3-dihydro-benzo[1,4]dioxine

2,3-Dihydro-benzo[1,4]dioxine-6-carbaldehyde (1 g, 6.09 mmol) was dissolved in acetonitrile (15 ml) (in a 50 ml round bottom flask), trimethylsulfoniumiodide (1.28 g, 6.28 mmol) and KOH (2.4 g) and some drops of water were added, and the resulting mixture was stirred for 1.5 hours at 60° C. The reaction mixture was concentrated by rotary evaporation. The residue was diluted in water and extracted with ethyl acetate. The organic layer was dried over Na₂SO₄, filtrated and concentrated. The residue was purified by flash chromatography (hexane/ethyl acetate 1:1) to give 1 g (100%) of the pure product.

¹H-NMR (CDCl₃): 6.80-6.77 (m, 3H); 4.27 (s, 4H); 3.78 (dd, J=2.61, 4.02, 1H); 3.11 (dd, J=4.02, 5.4, 1H); 2.79 (dd, J=2.61, 4.5, 1H)

### Synthesis of (R,S)-1-(2,3-Dihydro-benzo[1,4]dioxin-6-yl)-2-[4-(7-methoxy-naphthalen-1-yloxymethyl)-piperidin-1-yl]-ethanone

Lithium perchlorate (39.2 mg, 0.36 mmol) and potassium carbonate (101.9 mg, 0.73 mmol) were added to a solution of 4-(7-methoxy-naphthalen-1-yloxymethyl)-piperidine (100 mg, 0.36 mmol) and 6-oxiranyl-2,3-dihydro-benzo[1,4]dioxine (66 mg, 0.36 mmol) in DMF (1 ml). The reaction mixture was stirred over night at 80° C., concentrated by high vacuum, diluted in water and extracted with ethyl acetate. The organic layer was dried over Na₂SO₄ and concentrated. The product was purified by chromatography on silica gel (ethyl acetate) to give 62.5 mg (38%) as beige foam.

MS (ESI+) 450.5 [M+H⁺]

### Example 3

### (R,S)-6-{1-Hydroxy-2-[4-(7-methoxy-phthalazin-1-yloxymethyl)-piperidin-1-yl]-ethyl}-4H-benzo[1,4]oxazin-3-one

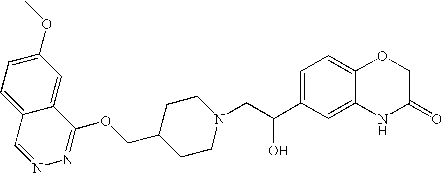

### Synthesis of 1-chloro-7-methoxy-phthalazine

A mixture of 7-methoxy-2H-phthalazin-1-one (2.2 g, 12.5 mmol, synthesised according to J. Am. Chem. Soc 1924, 1889) and POCl₃. (10 ml) was refluxed for 6 hours. The excess of POCl₃ was removed by rotary evaporation and the residue was diluted in ethyl acetate. The organic layer was washed with water and a bicarbonate solution, dried over MgSO₄ and concentrated. The product was purified by column chromatography (hexane/ethyl acetate 1:1).

¹H-NMR (CDCl₃): 9.33 (s, 1H); 7.92 (d, J=8.7 Hz, 1H); 7.58 (dd, J=8.7, 2.2 Hz, 1H); 7.52 (d, J=2.2 Hz, 1H); 4.0 (s, 3H)

MS (ESI+) 195/197 [M+H⁺]

### Synthesis of 4-(7-methoxy-phthalazin-1-yloxymethyl)-piperidin-1-carboxylic acid tert-butyl ester

NaH dispersion (55%, 96 mg) was added to a solution of 4-hydroxymethyl-piperidin-1-carboxylic acid tert-butyl ester (475 mg, 2.2 mmol) in DMF (10 ml) and stirred for 5 minutes. Then a solution of 1-chloro-7-methoxy-phthalazine (430 mg, 2.2 mmol) in DMF was added dropwise and the resulting reaction mixture was stirred for 4 hours at room temperature, afterwards it was diluted with ethyl acetate and water. The organic layer was washed with water, dried over MgSO₄ and concentrated. The product was purified by chromatography on silica gel (ethyl acetate) to give 709 mg (86%).

MS (ESI+) 374.5 [M+H⁺]

### Synthesis of 7-methoxy-1-(piperidin-4-ylmethoxy)-phthalazine

The BOC group was deprotected by TFA in dichloromethane according to example 1.

MS (ESI+) 284.5 [M+H⁺]

### Synthesis of 6-{2-[4-(7-methoxy-phthalazin-1-yloxy-methyl)-piperidin-1-yl]-acetyl}-4H-benzo[1,4]oxazin-3-one

Triethylamine (1 ml) was added to a mixture of 7-methoxy-1-(piperidin-4-ylmethoxy)-phthalazine (273 mg, 1 mmol) and 6-(2-chloro-acetyl)-4H-benzo[1,4]oxazin-3-one (225 mg, 1 mmol) in THF (5 ml) and heated for 2 hours at 50° C. A yellow precipitate was formed, which was filtrated and stirred in methanol/ethanol/THF to give 80 mg of the pure product.

MS (ESI+) 463.5 [M+H⁺]

### Synthesis of (R,S)-6-{1-hydroxy-2-[4-(7-methoxy-phthalazin-1-yloxymethyl)-piperidin-1-yl]-ethyl}-4H-benzo[1,4]oxazin-3-one

NaBH₄ (1 eq) was added to a solution of 6-{2-[4-(7-methoxy-phthalazin-1-yloxymethyl)-piperidin-1-yl]-acetyl}-4H-benzo[1,4]oxazin-3-one (40 mg) in ethanol (2 ml) and THF (2 ml) and stirred for 2 hours at room temperature. The reaction mixture was adsorbed on silica gel and purified by chromatography (dichloromethane/methanol 9:1+1% NH₄OH) to give 25 mg of the pure product.

MS (ESI+) 465.5 [M+H⁺]

### Example 4

### (R,S)-1-(2,3-Dihydro-benzo[1,4]dioxin-6-yl)-2-[4-(7-methoxy-phthalazin-1-yloxymethyl)-piperidin-1-yl]-ethanol

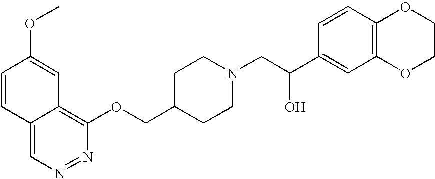

Lithium perchlorate (39.2 mg, 0.36 mmol) and potassium carbonate (101.9 mg, 0.73 mmol) were added to a solution of 7-methoxy-1-(piperidin-4-ylmethoxy)-phthalazine (100 mg, 0.36 mmol) and 6-oxiranyl-2,3-dihydro-benzo[1,4]dioxine (66 mg, 0.36 mmol) in DMF (1 ml). The reaction mixture was stirred over night at 80° C., concentrated with high vacuum, diluted in water and extracted with ethyl acetate. The organic layer was dried over Na₂SO₄ and concentrated. The product was purified by chromatography on silica gel (ethyl acetate) to give 58.7 mg (35%) as white foam.

MS (ESI+) 452.5 [M+H⁺]

### Example 5

### 6-{1-Hydroxy-2-[4-(7-methoxy-isoquinolin-1-yloxymethyl)-piperidin-1-yl]-ethyl}-4H-benzo[1,4]oxazin-3-one

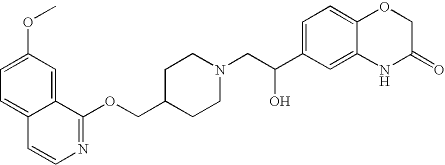

### Synthesis of 1-chloro-7-methoxy-isoquinoline

A mixture of 7-methoxy-2H-isoquinolin-1-one (6.5 g, 37 mmol, synthesised according to J. Heterocycl. Chem. 1985, 22, 328) and POCl₃ (50 ml) was refluxed for 6 hours. The excess of POCl₃ was removed by rotary evaporation and the residue was diluted in ethyl acetate. The organic layer was washed with ice cold water and bicarbonate solution, dried over MgSO4 and concentrated. The product was purified by column chromatography (hexane/ethyl acetate 3:1).

MS (ESI+) 194.5 [M+H⁺]

### Synthesis of 4-(7-methoxy-isoquinolin-1-yloxymethyl)-piperidin-1-carboxylic acid tert-butyl ester

NaH dispersion (55%, 240 mg) was added to a solution of 4-hydroxymethyl-piperidin-1-carboxylic acid tert-butyl ester (1075 mg, 5 mmol) in THF (20 ml) and stirred for 5 minutes. A solution of 1-chloro-7-methoxy-isoquinoline (965 mg, 5 mmol) in THF was added dropwise, and the resulting reaction mixture was stirred for 5 hours at 500C and over night at room temperature, afterwards it was diluted with ether and water. The organic layer was washed with water, dried over MgSO₄ and concentrated. The product was purified by chromatography on silica gel (hexane/ethyl acetate 3:1) to give 1.16 g (62%).

MS (ESI+) 373.5 [M+H⁺]

### Synthesis of 7-methoxy-1-(piperidin-4-ylmethoxy)-iso-quinoline

The BOC group was cleaved by TFA in dichloromethane according to example 1.

¹H-NMR (CDCl₃): 7.8 (d, J=5.97 Hz, 1H); 7.58 (d, J=8.91, 1H); 7.43 (d, J=2.52, 1H); 7.24, (dd, J=8.91, 2.52, 1H); 7.08 (d, J=5.97, 1H); 4.32 (d, J=6.51, 2H); 3.88 (s, 3H); 3.26-3.24 (m, 2H); 2.88-2.70 (m, 2H); 2.1-2.05 (m, 1H); 2.0-1.9 (m, 2H); 1.60-1.46 (m, 2H)

Synthesis of 6-{2-[4-(7-methoxy-isoquinolin-1-yloxy-methyl)-piperidin-1-yl]-acetyl}-4H-benzo[1,4]oxazin-3-one K₂CO₃ (1 eq) was added to a mixture of 7-methoxy-1-(piperidin-4-ylmethoxy)-isoquinoline (272 mg, 1 mmol) and 6-(2-chloro-acetyl)-4H-benzo[1,4]oxazin-3-one (225 mg, 1 mmol) in THF (5 ml) and stirred over night at 50° C. The reaction mixture was concentrated and the residue purified by chromatography on silica gel (ethyl acetate) to give 250 mg (54%) of the pure product.

MS (ESI+) 462.5 [M+H⁺]

### Synthesis of 6-{1-Hydroxy-2-[4-(7-methoxy-isoquinolin-1-yloxymethyl)-piperidin-1-yl]-ethyl}-4H-benzo[1,4]oxazin-3-one

NaBH₄ (40 mg) was added to a solution of 6-{2-[4-(7-methoxy-isoquinolin-1-yloxymethyl)-piperidin-1-yl]-acetyl}-4H-benzo[1,4]oxazin-3-one (200 mg, 0.5 mmol) in ethanol (20 ml) and stirred for 2 hours at room temperature. The reaction mixture was adsorbed on silica gel and purified by chromatography (dichloromethane/methanol 9:1+1% NH₄OH). The raw product was crystallised from ether to give 55 mg (28%) of the pure product.

MS (ESI+) 464 [M+H⁺]

### Example 6

### Synthesis of 1-(2,3-dihydro-benzo[1,4]dioxin-6-yl)-2-[4-(7-methoxy-isoquinolin-1-yloxymethyl)-piperidin-1-yl]-ethanol

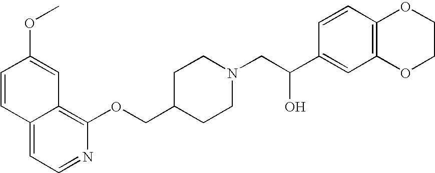

Lithium perchlorate (39.2 mg, 0.36 mmol) and potassium carbonate (101.9 mg, 0.73 mmol) were added to a solution of 7-methoxy-1-(piperidin-4-ylmethoxy)-isoquinoline (100 mg, 0.36 mmol) and 6-oxiranyl-2,3-dihydro-benzo[1,4]dioxine (66 mg, 0.36 mmol) in DMF (1 ml). The reaction mixture was stirred over night at 80° C., concentrated with high vacuum, diluted in water and extracted with ethyl acetate. The organic layer was dried over Na₂SO₄ and concentrated. The product was purified by chromatography on silica gel (ethyl acetate) to give 58.7 mg (35%) as white foam.

MS (ESI+) 451.5 [M+H⁺]

### Example 7

### 2-(3-{[(2,3-Dihydro-benzo[1,4]dioxin-6-yl-methyl)-amino]-methyl}-piperidin-1-yl)-1-(3-methoxy-quinolin-5-yl)-ethanol

### Synthesis of 3-azidomethyl-piperidine-1-carboxylic acid tert-butyl ester

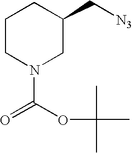

Triethylamine (2.6 ml, 18.6 mmol) and afterwards methanesulfonylchloride (0.8 ml, 10.3 mmol) were added dropwise to a solution of (3R)-hydroxymethyl-piperidin-1-carboxylic acid tert-butyl ester (2 g, 9.29 mmol, synthesised according to Tetrahedron Lett. 2002, 43, 8917 and Gazz. Chim. Ital. 1972, 102, 189) in dichloromethane (30 ml) at 0° C. The reaction mixture was stirred for 30 minutes at this temperature. Then sat. NaHCO₃ solution (20 ml) and dichloromethane (30 ml) were added. The two layers were separated and the organic layer was washed with brine (20 ml), dried over MgSO₄ and concentrated. The raw product was filtrated quickly through silica gel (hexane/ethyl acetate 1:1). The raw product was diluted in DMF (40 ml) and sodium azide (1.2 g, 18.4 mmol) was added. The reaction mixture was stirred for 5 hours at 80° C., concentrated by rotary evaporation and diluted with ether and water. The organic layer was dried over MgSO₄ and concentrated. The raw product was purified by chromatography on silica gel (hexane/ethyl acetate 4:1) to give 2.16 g (9 mmol) as oil.

MS (ESI+) 241.4 [M+H⁺]

### Synthesis of (R)-(2,3-dihydro-benzo[1,4]dioxin-6-yl-methyl)-piperidin-3-ylmethyl-amine

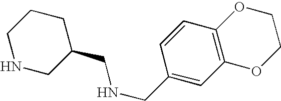

Polymer bound triphenylphosphine (6.3 g, 3.6 mmol/g) was added to a solution of 3-azidomethyl-piperidin-1-carboxylic adid tert-butyl ester (2.16 g, 9 mmol) in THF (60 ml) and water (1 ml). The mixture was stirred for 4 days at room temperature and then filtrated. The filtrate was concentrated and diluted in methanol (35 ml). 1,4-Benzodioxan-6-carboxaldehyde (1.48 g, 9 mmol) and 3A molecular sieve (9.6 g) were added. The reaction mixture was stirred for 5 hours at room temperature, then sodiumborohydride (1.2 g, 31.7 mmol) was added. The mixture was stirred for a further 16 hours at room temperature, concentrated and diluted in water (100 ml). The aqueous layer was extracted with dichloromethane (2×200 ml). The combined organic layers were dried over MgSO₄ and concentrated. The residue was purified by chromatography on silica gel (dichloromethane/methanol 19:1) to give 2.2 g of the product as oil. This oil was diluted in TFA (10 ml) and stirred for 1 hour. The mixture was concentrated, diluted in aqueous ammonia and extracted with dichloromethane (2×30 ml). The combined organic layers were dried over MgSO₄ and concentrated. 1.44 g (5.53 mmol) of the product could be isolated as oil.

MS (ESI+) 263.0 [M+H⁺]

### Synthesis of 2-bromo-1-(3-methoxy-quinolin-5-yl)-ethanone (Synthesis 2002, 83)

NBS (10.7 g, 60 mmol) was added to a solution of 3-bromoquinoline (10.4 g, 50 mmol) in conc. H₂SO₄ (50 ml) at room temperature and stirred over night. The reaction mixture was poured onto ice, then it was made alkaline with aqueous ammonia and extracted with ether. The organic layer was dried over MgSO₄ and concentrated. The product was purified by chromatography on silica gel (dichloromethane/hexane 6:4, dichloromethane, ethyl acetate) and recrystallised from methanol to give 8 g (56%) of 3,5-dibromoquinoline as white crystals.

1H-NMR (CDCl₃): 8.91 (d, J=2.2 Hz, 1H); 8.80 (d, J=2.2 Hz, 1H); 8.07 (d, J=7.8 Hz, 1H); 7.88 (d, J=7.8 Hz, 1H); 7.60 (t, J=7.8 Hz, 1H)

MS (ESI+) 285/287/289 [M+H⁺]

The above mentioned dibromide (2 mmol) was added to sodium methylate (4 mmol) in HMPT (8 ml) (Tetrahedron 2002, 58, 1125) and heated for 2 minutes at 90° C. in the microwave oven. This procedure was repeated 6 times. The combined reaction mixtures were poured onto water, extracted with ether, dried over MgSO₄ and concentrated. The product was purified by chromatography on silica gel (hexane/ethyl acetate 4:1) to give 2.78 g (67%) of the 5-bromo-3-methoxyquinoline.

This 5-bromo-3-methoxyquinoline was converted into 1-(3-methoxy-quinolin-5-yl)-ethanone as described in the literature (WO 0208224).

Br₂ (1 eq) and HBr (33% in acetic acid) were added to a solution of 1-(3-ethoxy-quinolin-5-yl)-ethanone (500 mg, 2.5 mmol) in acetic acid (10 ml). The mixture was stirred for 2 hours at room temperature. According to the MS a mixture of the mono- and the dibrominated product was formed. The reaction mixture was diluted with water and extracted with dichloromethane. The organic layer was washed with water and bicarbonate solution, dried over MgSO₄ and concentrated. The products were separated by chromatography on silica gel (hexane/ethyl acetate 2:1) to give 225 mg of the 2-bromo-1-(3-methoxy-quinolin-5-yl)-ethanone.

1H-NMR (CDCl₃): 8.75 (d, J=2.2 Hz, 1H); 8.65 (d, J=2.2 Hz, 1H); 8.33 (d, J=7.8 Hz, 1H); 8.13 (d, J=7.8 Hz, 1H); 7.64 (t, J=7.8 Hz, 1H); 4.65 (s, 2H); 4.01 (s, 3H).

MS (ESI+) 280/282 [M+H⁺]

### Synthesis of (1-RS)-2-(3(S)-{[(2,3-dihydro-benzo-[1,4]-dioxin-6-ylmethyl)-amino]-methyl}-piperidin-1-yl)-1-(3-methoxy-quinolin-5-yl)-ethanol

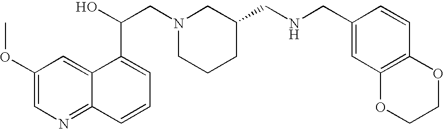

A solution of 2-bromo-1-(3-methoxy-quinolin-5-yl)-ethanone (0.113 g, 0.4 mmol) and (R)-(2,3-dihydro-benzo[1,4]dioxin-6-ylmethyl)-piperidin-3-ylmethyl-amine (0.106 g, 0.4 mmol) in THF (3 ml) was stirred over night at room temperature. The reaction mixture was concentrated and the residue dissolved in methanol (2 ml). After cooling to 0° C. NaBH₄ (0.031 g, 0.8 mmol) was added. The reaction mixture was stirred for one hour at 0° C. Afterwards water (3 ml) was added and then the reaction mixture was concentrated. The residue was purified by chromatography (dichloromethane/methanol 9:1 +1% NH₄OH) to give (1-RS)-2-(3(S)-{[(2,3-dihydro-benzo[1,4]dioxin-6-ylmethyl)-amino]-methyl}-piperidin-1-yl)-1-(3-methoxy-quinolin-5-yl)-ethanol (0.097 g, 0.21 mmol).

MS (ESI+) 464.5 [M+H⁺]

The following examples were prepared analogous to the above described:

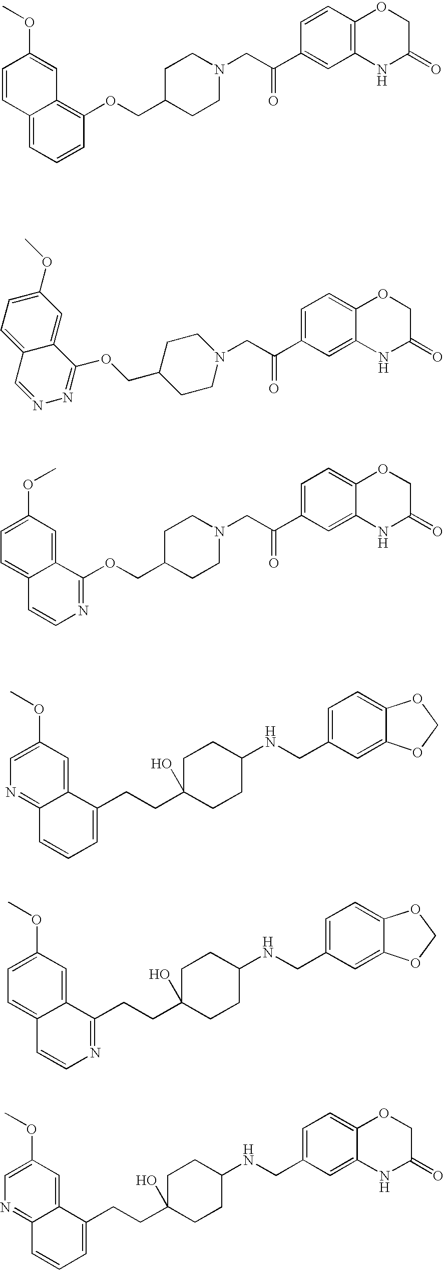

1. A compound of formula (1): 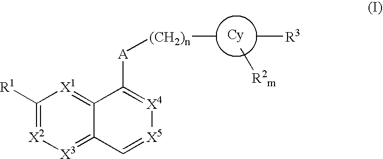
wherein
A is an oxygen or a sulphur atom, a NH, an alkylene, an alkenylene, an alkynylene or a heteroalkylene group,
X¹, X², X³, X⁴ and X⁵ are each independently of the others nitrogen atoms or groups of formula CH or CR⁴,
Cy is a cycloalkylene, a heterocycloalkylene, an arylene or a heteroarylene group,
R¹ is a hydrogen atom, a halogen atom, a hydroxy, an amino, a mercapto, an alkyl, a heteroalkyl, an alkyloxy, a heteroalkyloxy, a cycloalkyl, a heterocycloalkyl, an alkylcycloalkyl, a heteroalkylcycloalkyl, a cycloalkyloxy, an alkylcycloalkyloxy, a heterocycloalkyloxy or a heteroalkylcycloalkyloxy group,
the radicals R², each independently of any other(s), are a halogen atom, a hydroxy, an amino, a nitro or a mercapto group, an alkyl, an alkenyl, an alkynyl, a heteroalkyl, an aryl, a heteroaryl, a cycloalkyl, an alkylcycloalkyl, a heteroalkylcycloalkyl, a heterocycloalkyl, an aralkyl or a heteroaralkyl radical, or two of the radicals R² together form part of an aryl, heteroaryl, cycloalkyl, heterocycloalkyl, alkylcycloalkyl, heteroalkylcycloalkyl, aralkyl or a heteroaralkyl ring system,
R³ is an alkyl, alkenyl, alkynyl, heteroalkyl, aryl, heteroaryl, cycloalkyl, alkylcycloalkyl, heteroalkylcycloalkyl, heterocycloalkyl, aralkyl or heteroaralkyl radical,
R⁴ is a halogen atom, or a hydroxy, alkyl, alkenyl, alkynyl or heteroalkyl group,
n is 0, 1 or 2, and
m is 0, 1 or 2, or a pharmacologically acceptable salt, solvate, hydrate or a pharmacologically acceptable formulation thereof.

2. A compound according to claim 1, wherein A is an oxygen or a sulphur atom or a group of formula CH₂, CH₂CH₂, CH₂N(C₁-C₄-Alkyl), N(C₁-C₄-Alkyl)CH₂, CH₂O, OCH₂, CH₂S, SCH₂, CH₂CH(OH), CH(OH), CH(OH)CH₂, NHCO, CONH, C(═O)CH₂ or CH₂C(═O).

3. A compound according to claim 1, wherein three, four or five of the groups X¹, X², X³, X⁴ and X⁵ are CH groups.

4. A compound according to claim 1, wherein R¹ is a C₁-C₄alkyloxy or a C₁-C₄heteroalkyloxy group, wherein one or more hydrogen atoms of such groups may have been replaced by fluorine atoms.

5. A compound according to claim 1, wherein R¹ is a methoxy group.

6. A compound according to claim 1, wherein R² is a hydroxy, a C₁-C₄alkyl, a C₁-C₄heteroalkyl or a C₆-C₁₂heteroaralkyl group.

7. A compound according to claim 1, wherein R³ is a heteroalkylcycloalkyl or a heteroaralkyl group.

8. A compound according to claim 1, wherein R³ is a group of formula -B-Y, wherein B is an alkylene, an alkenylene, an alkynylene or a heteroalkylene group and Y is an aryl, a heteroaryl, an aralkyl, a heteroaralkyl, a cycloalkyl, a hetero-cycloalkyl, an alkylcycloalkyl or a heteroalkylcycloalkyl group.

9. A compound according to claim 8, wherein Y has one of the following structures, 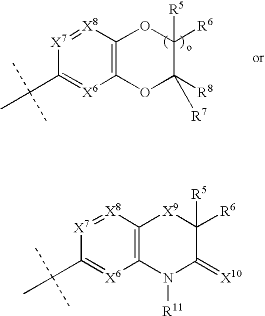
wherein X⁶, X⁷ and X⁸ are each independently of the others nitrogen atoms or groups of formula CR⁹, X⁹ and X¹⁰ are each independently of the others oxygen or sulphur atoms or groups of formula NR¹⁰, o is 0, 1 or 2, R⁵, R⁶, R⁷, R⁸ and R⁹ are each independently of the others hydrogen atoms, halogen atoms, hydroxy, alkyl, alkenyl, alkynyl or heteroalkyl groups and R¹⁰ and R¹¹ are each independently of the others hydrogen atoms, alkyl, alkenyl, alkynyl or heteroalkyl groups.

10. A compound according to claim 8, wherein Y has one of the following structures:

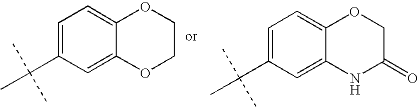

11. A compound according to claim 1, wherein the linker -A-(CH₂)ₙ— has a chain length of 2 or 3 atoms.

12. A compound according to claim 1, wherein R⁴ is a fluorine or a chlorine atom or a C₁-C₄alkyloxy or a C₃-C₆dialkylaminomethyl group wherein one or more hydrogen atoms of such groups may have been replaced by fluorine atoms.

13. A compound according to claim 1, wherein Cy is a cycloalkylene or a heterocycloalkylene group containing one or two rings and 4, 5, 6, 7, 8, 9 or 10 ring atoms.

14. A compound according to claim 1, wherein Cy has one of the following structures: 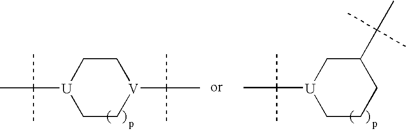
wherein U is a nitrogen atom or a group of formulas CH or COH and V is a nitrogen atom or a CH group and p is 0 or 1.

15. A pharmaceutical composition that comprises a compound according to claim 1 as active ingredient and, optionally, carrier substances and/or adjuvants.

16. (canceled)

17. A method of treating a subject suffering from or susceptible to a bacterial infection comprising administering to the subject a compound of claim 1.
